(19) United States
(12) Patent Application Publication
(10) Pub. No.: US 2008/0117066 A1
(43) Pub. Date: May 22, 2008

(54) Gas Monitoring Apparatus

(75) Inventors: Valery Alexandrovich Kononov, Weltevreden Park (ZA); Mathys Johannes De Beer, Boksburg (ZA)

Correspondence Address: QUARLES & BRADY LLP, 411 E. WISCONSIN AVENUE, SUITE 2040, MILWAUKEE, WI, 53202-4497, US

(73) Assignee: SCHAUENBURG FLEXADUX (PTY) LTD, Wadeville (ZA)

(21) Appl. No.: 10/562,752

(22) PCT Filed: Jun. 8, 2004
(86) PCT No.: PCT/IB04/01861

§ 371 Date: Nov. 6, 2006

(30) Foreign Application Priority Data
Jul. 4, 2003 (ZA) 2003/5203

(51) Int. Cl. G01N 33/00

(52) U.S. Cl. 340/632; 340/691.6

(57) ABSTRACT

Gas monitoring apparatus comprises a sensor module and an associated display module which can be physically connected together or separated. The sensor module contains gas sensors and is used to detect the concentration of gas, for example in a mine working. The sensor module transmits readings wirelessly to the display module, which includes a display to show the reading. Each sensor unit can communicate with multiple display units and vice versa.

### BACKGROUND OF THE INVENTION

This invention relates to gas monitoring apparatus which can be used, for example, in underground mines.

Gas monitoring apparatus is known which comprises a housing which supports one or more gas sensors, an electronic processing circuit, and a display. The apparatus is typically battery powered and portable.

A common use for such apparatus is to measure the concentration of gases in a mine working. Certain gases, such as methane, are lighter than air and tend to collect near the roof of a working. It is therefore common to mount the apparatus at the end of a pole and to hold it up near the roof to take a gas concentration reading. Under these circumstances, it can be difficult to read the display on the apparatus. If the apparatus has a memory function which stores the highest or most recent gas concentration reading on the display, a reading can be taken and the device brought back down from the roof to examine the display. However, this can lead to inaccurate readings as the gas concentration may vary from location to location.

It is an object of the invention to provide alternative gas monitoring apparatus.

### SUMMARY OF THE INVENTION

According to the invention there is provided gas monitoring apparatus comprising:
a first housing;
at least one gas sensor in the first housing;
measurement means in the first housing responsive to said at least one gas sensor to generate an output indicative of a measured gas concentration;
a transmitter in the first housing arranged to transmit signals indicative of the measured gas concentration;
a second housing;
a receiver in the second housing arranged to receive the signals indicative of the measured gas concentration; and
a display supported by the second housing for displaying the measured gas concentration.

Preferably, the first and second housings are connectable releasably together.

The transmitter in the first housing and the receiver in the second housing are preferably a radio transmitter and receiver.

The apparatus may be battery powered, by respective batteries in the first and second housings.

The batteries may be rechargeable, the first housing being provided with terminals receivable in a charger to charge the battery or batteries in both housings.

Preferably, the apparatus is arranged so that the battery or batteries in the first housing are charged simultaneously with the battery or batteries in the second housing when the two housings are connected together.

In a preferred embodiment of the invention, energy transfer means are provided on the respective housings to transfer sufficient energy from the first housing to the second housing to charge the battery or batteries in the second housing, without requiring electrical contact between the housings.

For example, a light source may be provided in the first housing, arranged to be activated when the first housing is received in a charger, and a photovoltaic cell may be provided on the second housing, the light source and the photovoltaic cell being located adjacent one another when the two housings are connected.

The sensor module may be arranged to transmit standard data signals to an associated display module and a broadcast signal to a plurality of display modules.

Preferably, the measurement means in the sensor module defines a gas concentration threshold, data signals indicating a gas concentration exceeding the gas concentration threshold being transmitted as broadcast signals.

The measurement means may be adjustable to permit the gas concentration threshold to be adjusted.

In a preferred embodiment, the sensor module can be designated as a master module that controls a communication protocol between itself and a plurality of display modules, or vice versa.

The display module preferably transmits the signals indicative of the measured gas concentration to a central reader.

### BRIEF DESCRIPTION OF THE DRAWINGS

FIG. 1 is a pictorial view of gas monitoring apparatus according to the invention;

FIG. 2 is a simplified schematic block diagram of the electronic circuitry of a receiver module of the apparatus;

FIG. 3 is a simplified schematic diagram of a battery charging circuit of the receiver module;

FIG. 4 is a simplified schematic diagram of the electronic circuitry of a sensor module of the apparatus;

FIG. 5 is a simplified schematic diagram of a battery charging circuit of the sensor module;

FIG. 6 is a pictorial view of a second embodiment of the gas monitoring apparatus according to the invention, showing the front face of the display module and the rear surface of the sensor module;

FIG. 7 is a flow chart showing the steps of operation of the display module; and

FIG. 8 is a flow chart showing the steps of operation of the sensor module.

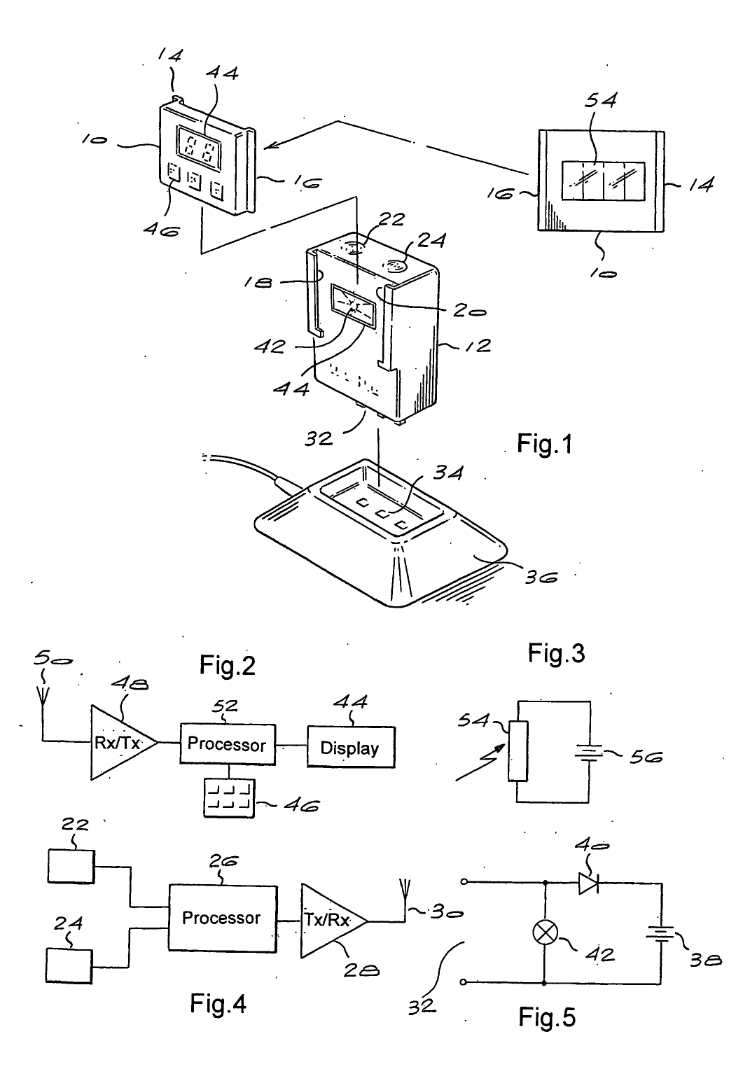

### DESCRIPTION OF AN EMBODIMENT

The illustrated gas monitoring apparatus comprises a display module 10 in a housing and a gas sensor module 12 in another housing. The two housings can be connected releasably together. The display module has a pair of outwardly extending flanges on opposed edges thereof, and the sensor module 12 has complemental inwardly extending flanges 18 and 20 on the front face thereof to allow the display module to be slid into position on the front face of the sensor module. The housings of the modules 10 and 12 will typically be moulded from a suitable plastics material.

The sensor module 12 has a pair of gas sensors 22 and 24 on an end surface thereof. For example, the sensors could be sensors for carbon monoxide (CO), oxygen (O₂) or (CH₄). Within the housing is a measurement circuit 26 which is typically microprocessor based, which provides an output related to the concentration of the relevant gases detected by the sensors. Such sensors and measurement circuits are well known as such and are therefore not discussed further in great detail.

Also within the housing of the sensor module 12 is a radio frequency transceiver 28 with an associated antenna 30. (see FIG. 4). A set of contacts 32 are provided at the base of the sensor module 12 which make contact with complemental contacts 34 in a battery charging unit 36.

The sensor module is battery powered, typically by means of nickel cadmium (NiCad) or nickel metal hydride (NiMh) batteries 38. When the sensor module is inserted into the charger 36, charging current is supplied to the battery 38 via a blocking diode 40. At the same time, a lamp or LED 42 is illuminated by the charging current.

The lamp 32 is mounted behind a transparent window 44 in the front face of the sensor module, between the flanges 18 and 20. The purpose of this arrangement is explained below.

The display module 10 supports a liquid crystal display (LCD) 44 on a front surface thereof, together with several push buttons 46 or other controls. The keypad 46 is used to enter operational and functional settings and can be used, for example, to set alarm thresholds for different gases. As indicated in FIG. 2, the display module includes a radio frequency transceiver 48 with an associated antenna 50, and a microprocessor based processing circuit 52 which controls the display 44.

On the rear surface of the display module 10 is photovoltaic cell 54 which is located so as to be adjacent the window 44 on the front face of the sensor module 12 when the two modules are connected together. The photovoltaic cell 54 is connected to a miniature rechargeable battery 56 which powers the display module, so that when sufficient light is incident on the photovoltaic cell, the battery 56 is charged.

Alternatively, the display module 10 can be charged through an additional or separate charging terminal either by way of the photovoltaic cell or by another charging circuit.

In either case, the sensor module and the display module are electrically isolated from one another, which provides an important safety feature in fiery mines or other dangerous environments where flammable gas may be present.

The apparatus can be operated with the display module and the sensor module connected together or physically separated. In either case, outputs from the gas sensor 22 and 24 are converted into gas concentration readings by the processing circuit 26 and a corresponding signal is transmitted wirelessly from the sensor module to the display module. This signal is received, processed and displayed on the LCD 44. Due to the wireless link between the two modules, the display module can be detached from the sensor module, allowing the sensor module to be held at the roof of a mine working on the end of a pole, for example, while the display module can be hand held and thus easily operated and observed by a user.

When the apparatus is placed in a charger with the two modules connected together, the lamp 42 illuminates the photovoltaic cell 54, which typically can generate an output current of 5 mA at about 5 V. This is sufficient to charge a miniature 3 V nickel metal hydride battery. The power consumption of the display is relatively low, requiring only a small battery.

By contrast, the relatively high capacity batteries required to operate the sensor module, which will typically be a 6 V, 2 Ah battery pack, can be charged directly from the charger. Thus, the versatility of the described arrangement is not compromised by the need for a special charging unit, separate chargers for the two modules, or the need to use disposable batteries in the display module.

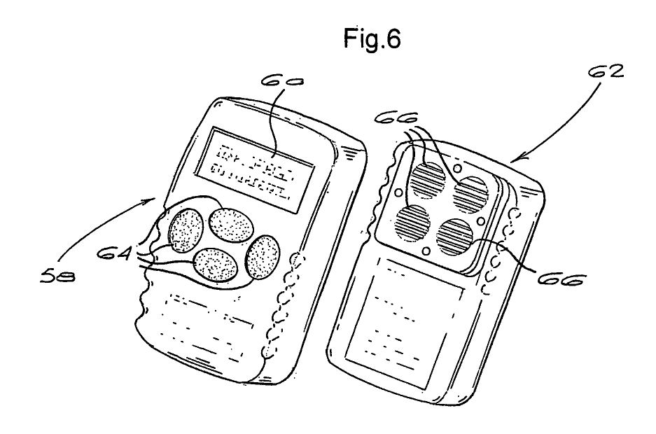

In FIG. 6, a second embodiment of the gas monitoring apparatus is shown. The display module 58 has a LCD 60 on its front face for displaying the signal received from the sensor module 62, and further has four push buttons 64 which are used to enter operational and functional settings to the gas monitoring apparatus. The sensor module 62 has four gas sensors 66 on its rear surface. The display unit 58 and sensor module 62 are mechanically connected to each other via complementally fitting tongue and groove formations (not shown) provided on the rear surface of the display module 58 and front face of the sensor module 62. To take the harsh underground conditions into account, these surfaces have also been provided with magnets, typically NdFeB magnets, to strengthen the mechanical connection between the display module 58 and sensor module 62.

In this embodiment of the invention, both the display module 58 and the sensor module 62 are battery powered, typically by means of individual NiMH batteries. A further feature of this embodiment is that the rear surface of the display module 58 and the front face of the sensor module 62 are manufactured from a semi-transparent material. A number of red light emitting diodes (LED's) are used as part of the internal circuitry of the modules, which LED's (not shown) are activated in an alarm situation, thereby providing a visual alarm to a user of the gas monitoring apparatus. This visual alarm will be visible to the user, whether the gas monitoring apparatus is used as an integrated unit, or as a separate display and sensor module.

In practice, it is quite likely that more than one apparatus according to the invention will be in use in the same general location. This could lead to interference between nearby instruments that could limit the rate of data transfer between respective display and sensor modules and thus delay the initiation of an alarm. In addition, multipath radio signal propagation in underground conditions can effectively limit communication speed and lead to data corruption. Preferably therefore, the transceivers 28 and 48 utilise spread spectrum communication techniques to ensure reliable transmission and reduction or elimination of interference with nearby apparatus. Data encryption is preferably also used.

Where a number of different display/sensor modules are used in relatively close proximity to one another, the possibility exists of sharing data between the modules. For example, a given sensor module can transmit data that is received by two or more display modules. In a preferred application, the sensor module has a user adjustable preset threshold, below which data is transmitted only to the associated display module. When a dangerous gas concentration is detected, exceeding the threshold level, the sensor module transmits a broadcast signal which can be received by all of the display modules within range, thus providing a general warning function. Such a broadcast signal can be characterised by greater signal strength than standard transmissions, or can be encoded with data identifying it as a broadcast message.

In certain situations, it may be desirable to use a single sensor module with multiple display modules, or a single display module with multiple sensor modules. In such a scenario, the single module can be programmed as a master module, with the multiple modules being programmed as slave modules, and a suitable communication protocol can be used to permit a single display module to interrogate the multiple sensor module, or for a single sensor module to communicate with multiple display modules.

In a typical application, the operation of the apparatus would be as follows.

When the apparatus is removed from the charger, the display module transmits a radio signal polling any nearby sensor modules. The nearest sensor module (typically, the sensor module mounted on the display module) identifies itself to the display module and a communication link between the two modules is established. The display module acts as a master unit and the sensor module acts as a slave unit.

In response to a data request signal issued every 200 milliseconds by the display module, the sensor module transmits measured data via a specified communication protocol. The received data is presented on the display of the display module and logged in memory.

Information logged in the display module can be downloaded via a radio link at the request of a third party. For example, a reader or a number of readers can request the transfer of logged data. Preferably, the reader or readers are located underground at strategic points in the mine. Alternatively, for example, the logged data can be transferred by a radio or infrared communication link when the unit is placed on the charger or a special charger/docking station. The transferred information can be displayed in a central control room, thereby providing quasi real time gas monitoring information for the whole mine.

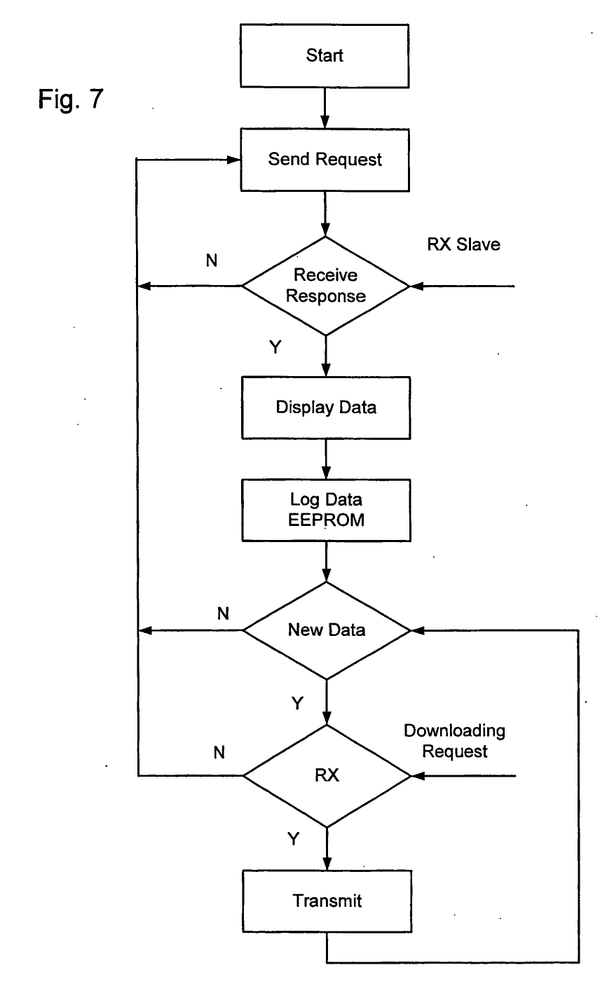

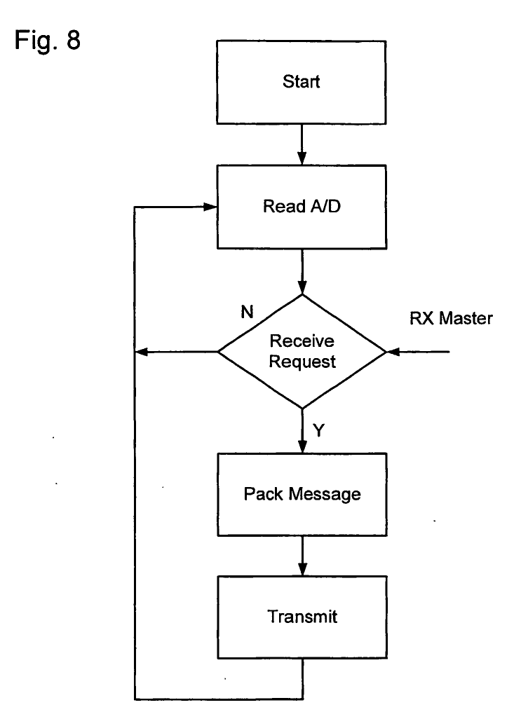

FIG. 7 is a flow chart showing the steps of operation of the steps of the display module, while FIG. 8 shows the steps of operation of the sensor module.

1-13. (canceled)

14. Gas monitoring apparatus comprising a sensor module and a display module,
the sensor module comprising:
a first housing;
at least one gas sensor in the first housing;
measurement means in the first housing responsive to said at least one gas sensor to generate an output indicative of a measured gas concentration; and
a wireless transmitter in the first housing arranged to transmit signals indicative of the measured gas concentration;
the display module comprising:
a second housing;
a wireless receiver in the second housing arranged to receive the signals indicative of the measured gas concentration; and
a display supported by the second housing for displaying the measured gas concentration,
wherein the first and second housings are connectable releasably together but are electrically isolated from one another, so that the apparatus can be operated with the display module and the sensor module connected together or physically separated.

15. Gas monitoring apparatus according to claim 14 wherein the transmitter in the first housing and the receiver in the second housing are a radio transmitter and receiver.

16. Gas monitoring apparatus according to claim 15 wherein the transmitter and receiver employ spread spectrum techniques.

17. Gas monitoring apparatus according to claim 14 which is battery powered, by respective batteries in the first and second housings.

18. Gas monitoring apparatus according to claim 17 wherein the batteries are rechargeable, at least the first housing being provided with terminals receivable in a charger to charge the battery or batteries in both housings.

19. Gas monitoring apparatus according to claim 18 which is arranged so that the battery or batteries in the first housing are charged simultaneously with the battery or batteries in the second housing when the two housings are connected together.

20. Gas monitoring apparatus according to claim 19 wherein energy transfer means are provided on the respective housings to transfer sufficient energy from the first housing to the second housing to charge the battery or batteries in the second housing, without requiring electrical contact between the housings.

21. Gas monitoring apparatus according to claim 20 wherein a light source is provided in the first housing, arranged to be activated when the first housing is received in a charger, and a photovoltaic cell is provided on the second housing, the light source and the photovoltaic cell being located adjacent one another when the two housings are connected.

22. Gas monitoring apparatus according to any one of claims 14 to 21 wherein the sensor module is arranged to transmit standard data signals to an associated display module and a broadcast signal to a plurality of display modules.

23. Gas monitoring apparatus according to claim 22 wherein the measurement means in the sensor module defines a gas concentration threshold, data signals indicating a gas concentration exceeding the gas concentration threshold being transmitted as broadcast signals.

24. Gas monitoring apparatus according to claim 23 wherein the measurement means is adjustable to permit the gas concentration threshold to be adjusted.

25. Gas monitoring apparatus according to claim 14 wherein either the sensor module can be designated as a master module that controls a communication protocol between itself and a plurality of display modules, or vice versa.

26. Gas monitoring apparatus according to claim 14 wherein the display module transmits the signals indicative of the measured gas concentration to at least one reader.
